(19) United States
(12) Patent Application Publication
(10) Pub. No.: US 2004/0087661 A1
(43) Pub. Date: May 6, 2004

(54) Treatment of CNS disorders with trans 4-3(3,4-dichlorophenyl)-1,2,3,4-tetrahydro-N-methyl-1-napthalenamine

(75) Inventors: Thomas P. Jerussi, Framingham, MA (US); Qun Kevin Fang, Wellesley, MA (US); Mark G. Currie, Sterling, MA (US)

Correspondence Address: HESLIN ROTHENBERG FARLEY & MESITI PC, 5 COLUMBIA CIRCLE, ALBANY, NY, 12203, US

(73) Assignee: Sepracor, Inc., Marlborough, MA

(21) Appl. No.: 10/662,997

(22) Filed: Sep. 16, 2003

Related U.S. Application Data
(60) Provisional application No. 60/411,303, filed on Sep. 16, 2002.

(51) Int. Cl. A61K 31/135

(52) U.S. Cl. 514/657

(57) ABSTRACT

Treatment of central nervous system disorders with (1R,4S)-trans 4-(3,4-dichlorophenyl)-1,2,3,4-tetrahydro-N-methyl-1-napthalenamine and with (1S,4R)-trans 4-(3,4-dichlorophenyl)-1,2,3,4-tetrahydro-N-methyl-1-napthalenamine is disclosed. A process for preparing 4-(3,4-dichlorophenyl)-1,2,3,4-tetrahydro-N-methyl-1-napthalenamine is also disclosed.

### CROSS REFERENCE TO RELATED APPLICATIONS

This application claims the priority of provisional application 60/411,303, filed Sep. 16, 2002, the entire disclosure of which is incorporated herein by reference.

### FIELD OF THE INVENTION

The present invention relates to methods of treating central nervous system disorders, and in particular, anxiety and eating disorders, as well as various other mental-related disorders, using (1R,4S)-trans 4-(3,4-dichlorophenyl)-1,2,3,4-tetrahydro-N-methyl-1-napthalenamine and (1S,4R)-trans 4-(3,4-dichlorophenyl)-1,2,3,4-tetrahydro-N-methyl-1-napthalenamine.

### BACKGROUND OF THE INVENTION

Clinicians recognize a distinction among central nervous system illnesses, and there have been many schemes for categorizing mental disorders. The Diagnostic and Statistical Manual of Mental Disorders, Fourth Ed., Text Revision, (hereinafter, the “DSM-IV-TR™”), published by the American Psychiatric Association, and incorporated herein by reference, provides a standard diagnostic system upon which persons of skill rely. According to the framework of the DSM-IV-TR™, the CNS disorders of Axis I include: disorders diagnosed in childhood (such as, for example, attention deficit disorder or “ADD” and attention deficit/hyperactivity disorder or “ADHD”) and disorders diagnosed in adulthood. CNS disorders diagnosed in adulthood include (1) schizophrenia and psychotic disorders; (2) cognitive disorders; (3) mood disorders; (4) anxiety related disorders; (5) eating disorders; (6) substance related disorders; (7) personality disorders; and (8) “disorders not yet included” in the scheme.

Of particular interest to the present invention are adult disorders of DSM-IV-TR™ categories (4) and (5). Anxiety related disorders of particular interest include agoraphobia, generalized anxiety disorder, phobic anxiety, obsessive-compulsive disorder (OCD), panic disorder, acute stress disorder, posttraumatic stress disorder, premenstrual syndrome, social phobia, chronic fatigue disorder, perimenopause, menopause and male menopause.

With respect eating disorders, of particular interest to the present invention are anorexia nervosa, bulimia nervosa, obesity and cachexia.

Other disorders of particular interest to the present invention include childhood/adolescence disorders exemplified by disruptive behavior disorders such as attention deficit disorder (ADD) and attention deficit/hyperactivity disorder (ADHD); substance abuse disorders exemplified by addiction to cocain, heroin, nicotine, alcohol, anxiolytic and hypnotic drugs, cannabis, amphetamines, hallucinogens, phenylcyclidine, volatile solvents and volatile nitrites; cerebral function disorders exemplified by dementia, Alzheimer's type dementia, Parkinson's disease, memory loss and autism; and disorders exemplified by urge and non-urge incontinence.

In general, treatment for psychoses, such as schizophrenia, for example, is quite different than treatment for mood disorders. While psychoses are treated with D₂ antagonists such as olanzapine (the “typical” and “atypical” antipsychotics), mood disorders are treated with drugs that inhibit the neuronal reuptake of monoamines, in particular, serotonin (5-HT), norepinephrine (NE) and dopamine (DA).

Common therapeutic agents for mood disorders include, but are not limited to, selective serotonin reuptake inhibitors (SSRI's), including fluoxetine, citalopram, nefazodone, fluvoxamine, paroxetine, and sertraline.

Sertraline, whose chemical name is (1S,4S)-cis 4-(3,4-dichlorophenyl)-1,2,3,4-tetrahydro-N-methyl-1-napthalenamine, is approved for the treatment of depression by the United States Food and Drug Administration, and is available under the trade name ZOLOFT® (Pfizer Inc., NY, N.Y., USA). The use of sertraline, (1R,4S)-trans 4-(3,4-dichlorophenyl)-1,2,3,4-tetrahydro-N-methyl-1-napthalenamine and (1S,4R)-trans 4-(3,4-dichlorophenyl)-1,2,3,4-tetrahydro-N-methyl-1-napthalenamine for the treatment of psychoses, psoriasis, rheumatoid arthritis and inflammation are disclosed in U.S. Pat. No. 4,981,870. The receptor pharmacology of the individual (1S,4R) and (1R,4S) enantiomers of trans 4-(3,4-dichlorophenyl)-1,2,3,4-tetrahydro-N-methyl-1-napthalenamine, is described by Welch et al., J. Med. Chem., 27:1508-1515 (1984).

### SUMMARY OF THE INVENTION

It has now been discovered that (1R,4S)-trans 4-(3,4-dichlorophenyl)-1,2,3,4-tetrahydro-N-methyl-1-napthalenamine (A); and (1S,4R)-trans 4-(3,4-dichlorophenyl)-1,2,3,4-tetrahydro-N-methyl-1-napthalenamine (B) are useful in the treatment of certain central nervous system disorders, including in particular, but not necessarily limited to: anxiety and eating disorders as herein described; disruptive behavior disorders, including ADD and ADHD; substance abuse and cerebral function disorders as herein described; and disorders characterized by non-urge and urge incontinence. A and B are also useful in the prophylaxis of migraine.

Compounds of the present invention are represented by formulae A and B:

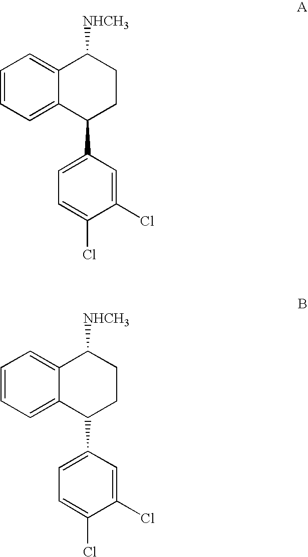

The graphic representations of racemic, ambiscalemic and scalemic or enantiomerically pure compounds used herein are taken from Maehr, J. Chem. Ed., 62:114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

In one aspect, the present invention relates to a method for treating anxiety and eating disorders, which involves the administration of a therapeutically effective amount of A or B, or a pharmaceutically acceptable salt thereof. Both anxiety and eating disorders are characterized in that symptoms of the disorders are reduced by increasing body monoamine levels, and in particular, body norepinephrine levels,

In another aspect, the invention relates to a process for preparing 4-(3,4-dichlorophenyl)-1,2,3,4-tetrahydro-N-methyl-1-napthalenamine, which involves:

(a) reacting 4-(3,4-dichlorophenyl)-3,4-dihydro-1-naphthalenone with an excess of formic acid and formamide to provide N-[4-(3,4-dichlorophenyl)-1,2,3,4-tetrahydronaphthalen- 1-yl]formamide; and

(b) reducing the N-[4-(3,4-dichlorophenyl)-1,2,3,4- tetrahydronaphthalen-1-yl]formamide with a hydride reducing agent, preferably, borane, thereby yielding 4-(3,4-dichlorophenyl)1,2,3,4-tetrahydro-N-methyl-1-napthalenamine.

### DETAILED DESCRIPTION OF THE INVENTION

The present invention provides several embodiments of a method for treating one or more CNS disorders, especially anxiety and eating disorders, and in the prophylaxis of migraine. The method encompasses administering a therapeutic amount of either pure A or pure B, or any mixture or pharmaceutically acceptable salt thereof.

Anxiety disorders treatable with the compounds of the invention include, but are not limited to: agoraphobia, generalized anxiety disorder, phobic anxiety, obsessive-compulsive disorder (OCD), panic disorder, acute stress disorder, posttraumatic stress disorder, premenstrual syndrome, social phobia, chronic fatigue disorder, perimenopause, menopause and male menopause.

Studies have shown that an increase in body monoamine levels, especially an increase in the level of norepinephrine, appears to reduce the symptoms associated with the aforementioned disorders. Thus, the compounds of the present invention are believed to provide their therapeutic activity by simultaneously blocking the reuptake of norepinephrine, serotonin and dopamine.

The compounds of formulae A and B are also effective for treating eating disorders. Eating disorders are defined as a disorder of one's appetite or eating habits or of inappropriate somatotype visualization. Eating disorders include, but are not limited to, anorexia nervosa, bulimia nervosa, obesity and cachexia.

Compounds of formulae A and B are also effective for treating disruptive behavior disorders, such as attention deficit disorder (ADD) and attention deficit disorder/hyperactivity (ADHD), which is in accordance with its accepted meaning in the art, as provided in the DSM-IV-TR™. These disorders are defined as affecting one's behavior resulting in inappropriate actions in learning and social situations. Although most commonly occurring during childhood, disruptive behavior disorders may also occur in adulthood.

Compounds of the present invention are also effective in the treatment of substance abuse disorders exemplified by addiction to cocaine, heroin, nicotine, alcohol, anxiolytic and hypnotic drugs, cannabis, amphetamines, hallucinogens, phenylcyclidine, volatile solvents and volatile nitrites; cerebral function disorders exemplified by dementia, Alzheimer's type dementia, Parkinson's disease, memory loss and autism; and disorders exemplified by urge and non-urge incontinence.

Compounds of the present invention are also effective in the prophylaxis of migraine.

Administration of compounds of the present invention results in a broad therapeutic profile and avoidance of side effects that are associated with an imbalance among the distribution of activity between norepinephrine, serotonin and dopamine receptors. Such side effects may include extrapyramidal symptoms, elevated serum prolactin levels, sexual dysfunction (decreased libido, anorgasmia, ejaculatory dysfunction), breast pain, weight gain and insomnia.

The term “treating” when used in connection with these disorders means amelioration, prevention or relief from the symptoms and/or effects associated with these disorders and includes the prophylactic administration of A or B, or a mixture or pharmaceutically acceptable salt thereof, to substantially diminish the likelihood or seriousness of the condition.

The magnitude of a prophylactic or therapeutic dose of A or B will vary with the nature and severity of the condition to be treated and the route of administration. The dose, and perhaps the dose frequency, will also vary according to the age, body weight and response of the individual patient. In general, the total daily dose ranges of A and B are from about 25 mg per day to about 1000 mg per day, preferably about 100 mg per day to about 600 mg per day, in single or divided doses.

It is further recommended that children, patients over 65 years old, and those with impaired renal or hepatic function, initially receive low doses and that the dosage by titrated based on individual responses and blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

Any suitable route of administration may be employed. For example, oral, rectal, intranasal, and parenteral (including subcutaneous, intramuscular, and intravenous) routes may be employed. Dosage forms include tablets, troches, dispersions, suspensions, solutions, capsules and patches.

Pharmaceutical compositions of the present invention include as active ingredient, a compound of formula A or formula B, or a mixture or pharmaceutically acceptable salt thereof, together with a pharmaceutically acceptable carrier, and optionally, with other therapeutic ingredients.

The term “pharmaceutically acceptable salt thereof” refers to salts prepared from pharmaceutically acceptable non-toxic acids including inorganic acids and organic acids. Exemplary acids that form pharmaceutically acceptable salts with the amines of the invention for use in the compositions of the present invention are acetic acid, benzenesulfonic (besylate) acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid and p-toluenesulfonic acid. The hydrochloric acid salt is particularly preferred.

Compositions suitable for oral, rectal, and parenteral administration are encompassed by the present invention. A preferred route of administration is oral. The compositions may be conveniently presented in unit dosage form and prepared by any of the methods well known in the art of pharmacy. Preferred unit dosage formulations are those containing an effective dose, or an appropriate fraction thereof, of the active ingredients.

The compositions of the present invention also include a pharmaceutically acceptable carrier. The carrier may take a wide variety of forms, depending on the forms preparation desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by a standard aqueous or nonaqueous techniques. Oral and parenteral sustained release dosage forms may also be used.

Oral syrups, as well as other oral liquid formulations, are well known to those skilled in the art, and general methods for preparing them are found in any standard pharmacy school textbook, for example Remington: The Science and Practice of Pharmacy. Chapter 86 of the 19th edition of Remington entitled “Solutions, Emulsions, Suspensions and Extracts” describes in complete detail the preparation of syrups (pages 1503-1505) and other oral liquids.

Similarly, sustained release formulation is well known in the art, and Chapter 94 of the same reference, entitled “Sustained-Release Drug Delivery Systems,” describes the more common types of oral and parenteral sustained-release dosage forms (pages 1660-1675.) The relevant disclosure of each of these chapters is incorporated herein by reference. Because they reduce peak plasma concentrations, as compared to conventional oral dosage forms, controlled release dosage forms are particularly useful for providing therapeutic plasma concentrations while avoiding the side effects associated with high peak plasma concentrations that occur with conventional dosage forms.

Preparation of the compounds of the present invention is illustrated below in Scheme 1 and its accompanying narrative.

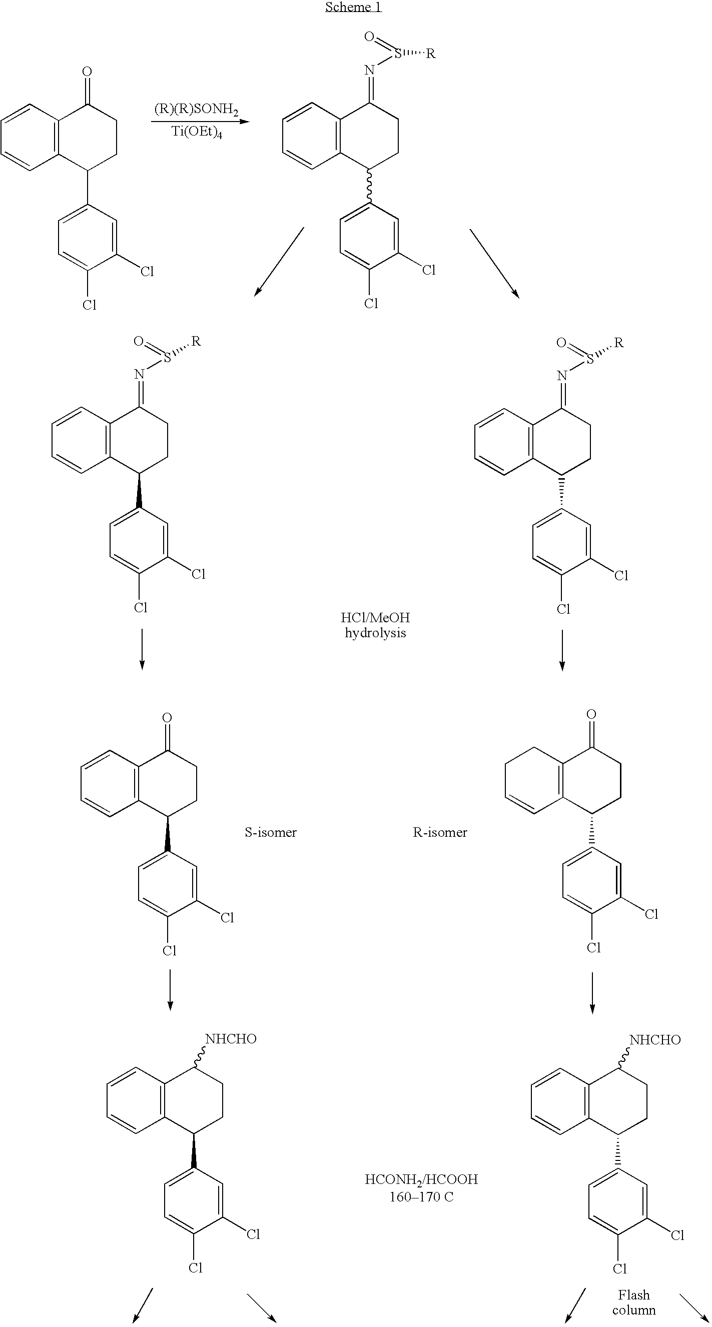

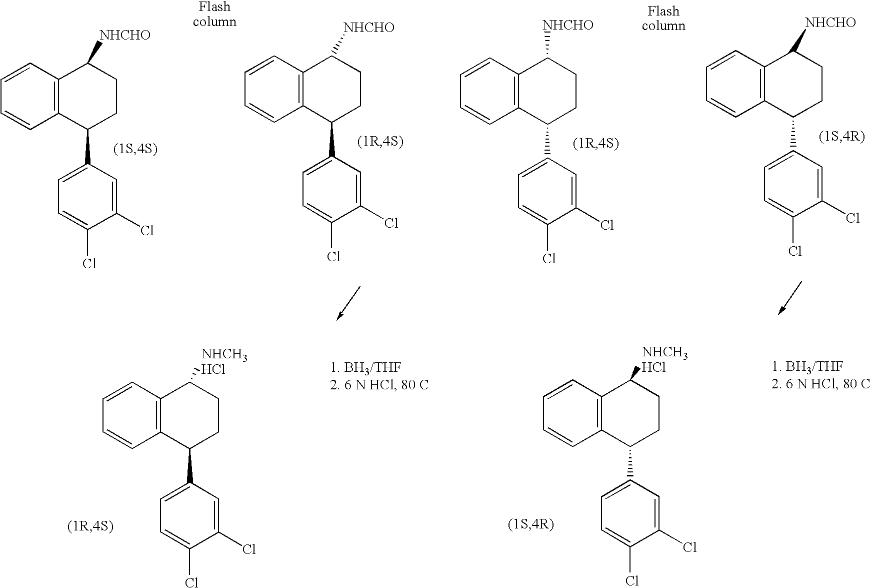

In the compound

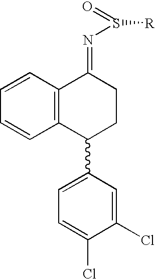

of Scheme 1, R is

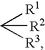

wherein R¹, R² and R³ are each independently alkyl. In a preferred embodiment of the compounds, R is t-butyl.

Synthesis of 2-methyl-propane-2-sulfinic acid [4-(3,4-dichloro phenyl)-1,2,3,4-tetrahydro-naphthalen-y-yl]-amide (tetralone t-butanesulfinimine):

To a solution of 4-((3,4-dicholorophenyl)-3,4-dihydro-1-naphthalenone (12 g) in THF (40 mL) was added (R)-t-butanesulfinamide (5.2 g) and Ti(OEt)₄ (85 mL 20%) in EtOH. The reaction mixture was heated to 60° C. for 13 h. The reaction mixture was cooled to rt, and poured to a brine solution (100 mL) with stirring. The suspension was then added EtOAc (300 mL) and stirred to 10 min. The suspension was filtered and the filtrate was concentrated to ca 50 mL. It was then added EtOAc (100 mL), the organic phase was then separated and concentrated to give a crude reaction mixture. The final products were isolated from the crude products by careful flash column using EtOAc and hexane (3:7 to 1:1) to give ca 3 g starting ketone, and (1R,4S)-4-(3,4-dichlorophenyl)-3,4-dihydro-1-naphthalenone tert-butanesulfinimine (2.5 g, first product) as an oil that solidified on standing. ¹H NMR (CDCl₃) δ 1.33 (S, 9H), 2.10-2.20 (m, 1H), 2.28-2.38 (m, 1H) 2.88-2.98 (m, 1H), 3.34-3.44 (m 1H), 4.12-4.24 (m, 1H), 6.84-6.88 (m, 2H), 7.20 (s, 1H), 7.25-7.40 (m, 3H), 8.22-8.28 (m, 1H). The other product (1R,4R)-4-(3,4-dichloro phenyl)-3-4-dihydro-1-naphthalenone t-butanesulfinimine (3.0 g, second product, lower R_{f}) was isolated also as oil that solidified on standing. ¹H NMR (CDCl₃) δ 1.34 (S, 9H), 2.05-2.18 (m, 1H), 2.28-2.38 (m, 1H), 3.15-3.25 (m, 2H), 4.16-4.22 (m, 1H), 6.84-6.88 (m, 2H), 7.20 (s,1H), 7.25-7.40 (m, 3H), 8.22-8.28 (m, 1H).

Synthesis of (R)-4-(3,4-dichlorophenyl)-3,4-dihydro-1-naphthalenone:

(1R,4R)-4-(3,4-dichlorophenyl)3,4-dihydro-1-naphthalenone t-butanesulfinimine (3.0 g, second product) was dissolved in MeOH (20 mL) and concentrated HCl (4 mL) at rt. The reaction mixture was stirred at rt to give a suspension. It was filtered and the solids were washed with hexane to give 1.2 g product. The enantiomeric purity was determined to be >99.3% by HPLC analysis with a ChiralPak AS 10 μm, 4.6×250 mm, Hexane/IPA (90:10), UV 220 nm, R-isomer 8.23 min. S-isomer 12.25 min. ¹H NMR (CDCl₃) δ 2.20-2.32 (m, 1H), 2.42-2.53 (m, 1H) 2.57-2.78 (m, 2H), 4.28 (dd=4.6, 8.1 Hz, 1H), 6.95 (dd, J=2.1, 7.6 Hz, 2H), 7.23 (d J=2.0 Hz, 1H), 7.37-50 (m, 3H), 8.13 (d, J=7.6 Hz, 1H). [α]=−66° (c=1, acetone).

Synthesis of (S)-4-(3,4-dichlorophenyl)-3,4-dihydro-1-naphthalenone.

The previous procedure was used, starting from (1R,4S)-4-(3,4-dichlorophenyl)-3,4-dihydro-1-naphthalenone t-butanesulfinimine. 1.7 g of product (>99% ee) was obtained. [α]=+62, c=1, acetone). ¹H NMR spectrum of the product is the same as that of its enantiomer.

Synthesis of (1S,4R) and (1R,4R)-N-[4-(3,4-dichlorophenyl)-1,2,3,4-tetrahydro-naphthalen-1-yl]-formamide:

(R)-4-(3,4-dichlorophenyl)-3,4-dihydro-1-naphthalenone (1.2 g) was added formic acid (3 mL) and formamide (3 mL). The reaction mixture was heated to 160-165° C. for 15 h under nitrogen atmosphere. The reaction mixture was cooled to rt and decanted the solvent. The residue solids was passed through flash column using EtOAc:Hexane (3:7 to 1:1) to give and (1R,4R)-formamide (400 mg, first spot), and (1S,4R)-formamide (360 mg). ¹H NMR of the first product [(1R,4R)-isomer]: (CDCl₃) δ 1.80-2.10 (m, 3H), 2.10-2.20 (m, 1H), 4.00-4.10 (m, 1H), 5.22-5.30 (m, 1H), 6.10-6.20 (m, 1H), 6.80-6.90 (M, 1H), 6.90-6.96 (m, 1H), 7.10-7.40 (m, 5H), 8.22 (s, 1H). M+320. ¹H NMR of the second product [(1S,4R)-isomer: δ 1.64-1.90 (m, 2H), 2.10-2.28 (m, 2H), 4.10 (m, 1H), 5.38-5.42 (m, 1H), 5.82-6.05 (m, 1H), 6.80-6.90 (m, 2H), 7.10-40 (m, 5H), 8.28 (s, 1H). Mass Spec. M⁺ 320.

Synthesis of (1S*,4R*)-trans 4-(3,4-dichlorophenyl)-1,2,3,4-tetrahydro-N-methyl-1-napthalenamine HCl (racemic mixture of A and B HCl):

(1S*,4R*) formamide (1.0 g) was dissolved in THF (7 mL), and added BH₃ THF (1M, 9.3 mL, 3 eq. The reaction mixture was heated to 75-80° C. for 3 h and stirred at rt overnight. The reaction mixture was quenched with MeOH (20 mL). The mixture was concentrated to give a residue, which was dissolved in 10% HCl (20 mL). The solution was heated to 80-90° C. for 9 h, and basified with potassium carbonate, and extracted with EtOAc (25 mL). The organic phase was separated and washed with water, brine, dried over Na₂SO₄. Concentrated to give the free base. It was converted to its HCl salt in TBME with HCl/Et2O to give the product (0.75 g). ¹H MNR (CD₃OD) δ 1.86-1.96 (m, 1H), 2.04-2.12 (m, 1H), 2.18-2.28 (m, 1H), 2.30-2.42 (m, 1H), 2.78 (s, 3H), 4.34 (m, 1H), 4.60 (m, 1H), 6.93-7.00 (m, 2H), 7.15 (s, 1H), 7.34-7.44 (m, 3H), 7.57-7.59 (d, J=7.2 Hz, 1H). Mass Spec. M⁺ 305.

Synthesis of (1S,4R)-trans 4-(3,4-dichlorophenyl)-1,2,3,4-tetrahydro-N-methyl-1-napthalenamine HCl by Resolution with (S)-Mandelic Acid:

Racemic trans-4-(3,4-dichlorophenyl)-1,2,3,4-tetrahydro-N-methyl- 1-napthalenamine (3 g) was dissolved in anhydrous ethanol (30 g) and added (S)-mandelic acid (1.5 g). The reaction mixture was heated to reflux for 30 min. and cooled to rt. The reaction solution was concentrated to give oil (ca 3 mL ethanol left). To it was added EtOAc (30 mL) and stirred for 1 h at rt. The solid formed from the solution was collected by filtration and dried (1.73 g). The solid was dissolved in hot EtOAc (35 mL), and cooled to rt in 30 min, and stirred for 1 h. The solid was collected by filtration and dried to give (1S,4R)-4-(3,4-dichlorophenyl)-1,2,3,4-tetrahydro-N-methyl-1-napthalenamine-(S)-mandelate (1.3 g). Ee of the product was >99% by HPLC. The solid (1.1 g) was converted to its free base with potassium carbonate, and treated with HCl/ether in MeOH to give the HCl salt (0.73 g). ¹H NMR spectrum was identical to its racemate. (1R,4S)-4-(3,4-dichlorophenyl)-1,2,3,4-tetrahydro-N-methyl-1-napthalenamine HCl was prepared from the mother liquor, after enriched with (R)-mandelic acid. Mass Spec M⁺ 305.

The commercial form of sertraline [(S,S)-cis] and its isomeric analogues were tested for their inhibition of functional uptake of serotonin (5-HT), norepinephrine (NE), or dopamine (DA), in synaptosomes prepared from rat whole brain, hypothalamus, or corpus striatum, respectively. Compounds were tested initially at 10 μM in duplicate, and if ≧50% inhibition of uptake was observed, they were tested further at 10 different concentrations in duplicate in order to obtain full inhibition curves. IC₅₀ values (concentration inhibiting control activity by 50%) was then determined by nonlinear regression analysis of the inhibition curves and tabulated below.

### Experimental Conditions for Monoamine Uptake Assays

Serotonin Functional Uptake Assay

Characterization of serotonin uptake is performed using synaptosomes isolated in a 0.32 M sucrose buffer from a male Wistar rat cortex. The uptake of radiolabelled serotonin by synaptosomes (100 μg of proteins/point) is allowed by incubating them in a deep well for 15 minutes at 37° C. in presence of test compounds and [3H]5-hydroxytryptamin (0.1 μCi/point).

Synaptosomes and [³H]5-hydroxytryptamine are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 15 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [³H]5-hydroxytryptamine. The radioactivity associated to the synaptosomes retained onto the unifilter corresponding to the uptake is then measured with a microplate scintillation counter Topcount, Packard using a scintillation liquid microscint 0, Packard.

The reference compound is imipramine tested at 10 concentrations ranging from 10⁻¹¹ M to 10⁻⁵ M in order to obtain an IC₅₀ value. See, Perovics and Müller, “Pharmacological profile of hypericum extract: effect on serotonin uptake by postsynaptic receptors,” Arzeim. Forsch./Drug Res., 45:1145-1148 (1995).

Dopamine Functional Uptake Assay

Characterization of dopamine uptake is performed using synaptosomes isolated at Cerep in a 0.32 M sucrose buffer from a male Wistar rat striatum. The uptake of radiolabelled dopamine by synaptosomes (20 μg of proteins/point) is allowed by incubating them in a deep well for 15 minutes at 37° C. in presence of test compounds and [³H]-dopamine (0.1 μCi/point).

Synaptosomes and [³H]-dopamine are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 15 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [³H]-dopamine. The radioactivity associated to the synaptosomes retained onto the unifilter corresponding to the uptake is then measured with a microplate scintillation counter Topcount, Packard using a scintillation liquid microscint 0, Packard. The reference compound is GRB12909 tested at 8 concentrations ranging from 10⁻¹¹ M to 10⁻⁶ M in order to obtain an IC₅₀ value. Jankowsky et al., “Characterization of sodium-dependent [³H] GBR-12935 binding in brain: a radioligand for selective labeling of the dopamine transport complex,” J. Neurochem., 46:1272-1276 (1986).

Norepinephrine Functional Uptake Assay

Characterization of norepinephrine uptake is performed using synaptosomes isolated at Cerep in a 0.32 M sucrose buffer from a male Wistar rat hypothalamus. The uptake of radiolabeled norepinephrine by synaptosomes (100 μg of proteins/point) is allowed by incubating them in a deep well for 20 minutes at 37° C. in presence of test compounds and [³H]-norepinephrine (0.1 μCi/point).

Synaptosomes and [³H]-norepinephrine are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 20 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [³H]-norepinephrine. The radioactivity associated to the synaptosomes retained onto the unifilter corresponding to the uptake is then measured with a microplate scintillation counter Topcount, Packard using a scintillation liquid microscint 0, Packard.

The reference compound is imipramine tested at 13 concentrations ranging from 10⁻¹¹ M to 10^{31 5} M in order to obtain an IC₅₀ value. See, Perovics and Müller, “Pharmacological profile of hypericum extract: effect on serotonin uptake by postsynaptic receptors,” Arzeim. Forsch./Drug Res., 45:1145-1148 (1995).

TABLE 1
IC₅₀ Values (μM) for Sertraline and Analogues in
Functional Monoamine Uptakes Assays

| | 5-HT | NE | DA |
|---|---|---|---|
| sertraline | 0.0016 | 0.31 | 0.048 |
| (R,R) cis | 0.11 | 0.11 | 0.039 |
| A* | 0.0075 | 0.012 | 0.0046 |
| B** | 0.33 | 0.024 | 0.026 |
| A + B | 0.0070 | 0.0056 | 0.0073 |
| imipramine | 0.054/0.051 | — | — |
| protriptyline | — | 0.0036 | — |
| GBR 12909 | — | — | 0.0028/0.0051/0.0034 |

*A (1R,4S)-trans 4-(3,4-dichlorophenyl)-1,2,3,4-tetrahydro-N-methyl-1-napthalenamine
**B (1S,4R)-trans 4-(3,4-dichlorophenyl)-1,2,3,4-tetrahydro-N-methyl-1-napthalenamine
/ separates multiple determinations
— <50% inhibition

The IC₅₀ value for (R,R) had to be estimated because the lowest plateau of the inhibition curve (corresponding to 100% inhibition) was not reached at the highest concentration tested or 100% or control activity was not apparent with the lowest concentration.

As shown in Table 1, A and B exhibit similar inhibitory potency on the neuronal uptake of NE, DA, and 5HT. Currently, the therapeutic approach to treating affective disorders in man is the selective inhibition of a single monoamine uptake mechanism or the dual inhibition of two of these molecular targets. The equipotent inhibition of neuronal uptake of NE, DA and 5HT provides the clinician with the ability to more effectively treat the disorders mentioned specifically herein by elevating all of the monoamine levels in the brain simultaneously and over the same dose-range without the need to titrate separate drugs.

Exemplary pharmaceutical formulations of the present invention include:

Tablets - Composition per dosage unit

| | |
|---|---|
| A or B | 25 mg |
| croscarmellose | 60 mg |
| colloidal silicon dioxide | 8 mg |
| magnesium stearate | 1 mg |
| microcrystalline cellulose | 190 mg |
| croscarmellose | 15 mg |
| talc | 10 mg |
| Total | 534 mg |

The A or B and silicon dioxide are dry mixed, the first portion of croscarmellose is added and the mixture is further dry mixed. The magnesium stearate is added, dry mixed and the mixture is run through a roller compactor and mill. The resulting dry granulate is mixed with the remaining three ingredients and compressed into tablets.

Powder-filled Capsules - Composition per unit dosage

| A or B | 200 mg |
|---|---|
| lactose | 250 mg |
| corn starch | 60 mg |
| magnesium stearate | 5 mg |
| Total | 515 |

The (A) or (B), lactose and cornstarch, in the proportions shown above, are blended until uniform and then the magnesium stearate is blended into the resulting powder, which is sieved and filled into suitably sized, two-piece, hard gelatin capsules using conventional machinery. Other doses may be prepared by altering the fill weight and, if necessary, changing the capsule size to suit.

1. A method for treating an anxiety disorder in a human, the method comprising administering to a person in need of therapy for an anxiety disorder, a therapeutically effective amount of a compound chosen from (1R,4S)-trans 4-(3,4-dichlorophenyl)-1,2,3,4-tetrahydro-N-methyl-1-napthalenamine (A); (1S,4R)-trans 4-(3,4-dichlorophenyl)-1,2,3,4-tetrahydro-N-methyl-1-napthalenamine (B); 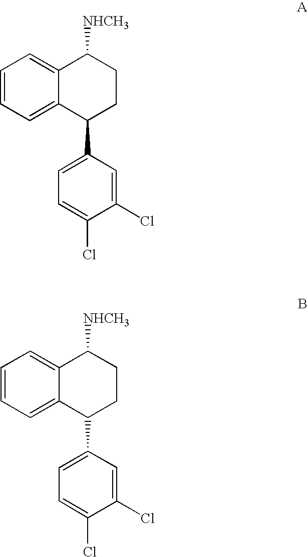
mixtures of A and B; and pharmaceutically acceptable salts thereof.

2. The method according to claim 1, wherein the anxiety disorder is chosen from agoraphobia, generalized anxiety disorder, phobic anxiety, obsessive-compulsive disorder (OCD), panic disorder, acute stress disorder, posttraumatic stress disorder, premenstrual syndrome, social phobia, chronic fatigue disorder, perimenopause, menopause and male menopause.

3. A method for treating an eating disorder in a human, the method comprising administering to a person a need of therapy for an eating disorder, a therapeutically effective amount of a compound chosen from (1R,4S)-trans 4-(3,4-dichlorophenyl)-1,2,3,4-tetrahydro-N-methyl-1-napthalenamine (A); (1S,4R)-trans 4-(3,4-dichlorophenyl)-1,2,3,4-tetrahydro-N-methyl-1-napthalenamine (B); 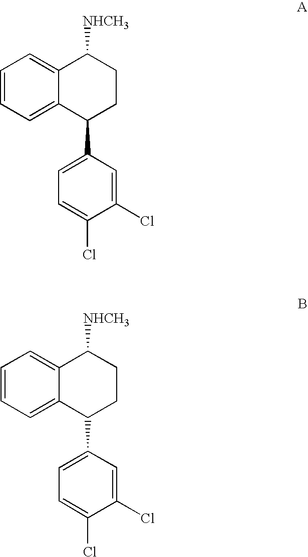
mixtures of A and B; and pharmaceutically acceptable salts thereof.

4. The method according to claim 3, wherein the eating disorder is chosen from anorexia nervosa, bulimia nervosa, obesity and cachexia.

5. A method for treating a condition chosen from disruptive behavior disorders, substance abuse and cerebral function disorders and disorders characterized by non-urge and urge incontinence in a human, the method comprising administering to a person in need of therapy for the chosen condition, a therapeutically effective amount of a compound chosen from (1R,4S)-trans 4-(3,4-dichlorophenyl)-1,2,3,4-tetrahydro-N-methyl-1-napthalenamine (A); (1S,4R)-trans 4-(3,4-dichlorophenyl)-1,2,3,4-tetrahydro-N-methyl-1-napthalenamine (B); 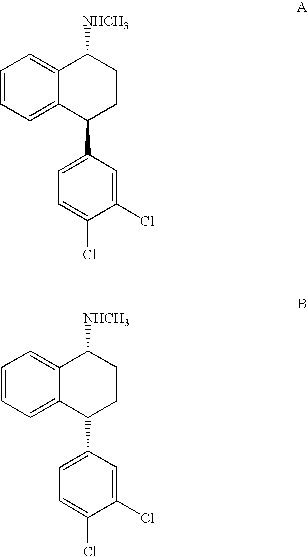
mixtures of A and B; and pharmaceutically acceptable salts thereof.

6. A method for the prophylaxis of migraine in a human, the method comprising administering to a person at risk or in need of therapy for a migraine, a therapeutically effective amount of a compound chosen from (1R,4S)-trans 4-(3,4-dichlorophenyl)-1,2,3,4-tetrahydro-N-methyl-1-napthalenamine (A); (1S,4R)-trans 4-(3,4-dichlorophenyl)-1,2,3,4-tetrahydro-N-methyl-1-napthalenamine (B); 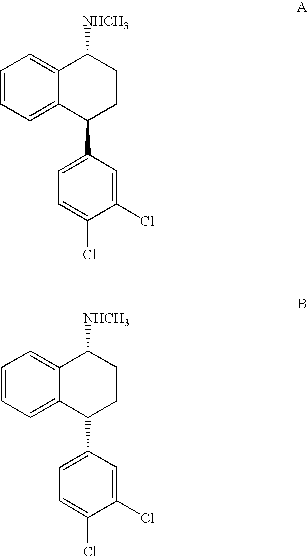
mixtures of A and B; and pharmaceutically acceptable salts thereof.

7. A process for preparing 4-(3,4-dichlorophenyl)-1,2,3,4-tetrahydro-N-methyl-1-napthalenamine comprising:
(a) reacting 4-(3,4-dichlorophenyl)-3,4-dihydro-1-naphthalenone with an excess of formic acid and formamide to provide N-[4-(3,4-dichlorophenyl)-1,2,3,4-tetrahydronaphthalen- 1-yl]formamide; and
(b) reducing the N-[4-(3,4-dichlorophenyl)-1,2,3,4-tetrahydronaphthalen-1-yl]formamide with a hydride reducing agent.

8. The process according to claim 7, wherein the hydride reducing agent is borane.

9. A compound of the formula: 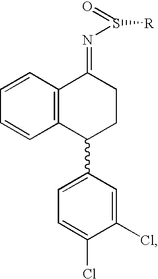
wherein R is 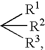
and R¹, R² and R³ are each independently alkyl.

10. A compound according to claim 9, wherein R is t-butyl.
